(19) United States
(12) Patent Application Publication
(10) Pub. No.: US 2026/0284292 A1
(43) Pub. Date: Sep. 24, 2026

(54) Devices And Methods For Providing An Anticlotting Element For Use With A Medical Waste Collection System

(71) Applicant: Stryker Corporation, Portage, MI (US)

(72) Inventors: Clayton H. Meyers, Paw Paw, MI (US); Michael Zollinger, Chelsea, MI (US)

(73) Assignee: Stryker Corporation, Portage, MI (US)

(21) Appl. No.: 19/474,554

(22) PCT Filed: Apr. 11, 2024
(86) PCT No.: PCT/US2024/023956

§ 371 Date: Oct. 10, 2025

Related U.S. Application Data
(60) Provisional application No. 63/458,502, filed on Apr. 11, 2023.

(51) Int. Cl. A61M 1/00

(52) U.S. Cl. CPC A61M 1/79; A61M 1/63; A61M 2205/02; A61M 2205/7563

(57) ABSTRACT

Devices and methods for providing an anticlotting element for use with a medical waste collection system. A manifold includes a trunk, a head, at least one inlet fitting, and the anticlotting element. The head defines a cavity, and the anticlotting element may be disposed within the cavity of the head. The anticlotting element may be positioned, in elevation, between backflow prevention valves that are coupled to the head. The anticlotting element may be disposed within the trunk, such as within a filter element, within a first leg of the trunk, and/or within a second leg of the trunk. An anticlotting assembly may be provided and configured to be coupled to suction tubes and/or to a manifold. The anticlotting element may be mixed with detergent configured to be prefilled to an upper waste container of the medical waste collection system.

### PRIORITY CLAIM

This application claims priority to and all the benefits of U.S. Provisional Patent Application No. 63/458,502, filed Apr. 11, 2023, the entire contents of which are hereby incorporated by reference.

### BACKGROUND

A byproduct of some surgical procedures is the generation of liquid, semisolid, and/or solid waste material. The liquid waste material may include bodily fluids and irrigating solution(s) at the surgical site, and the solid and semisolid waste material may include bits of tissue and pieces of surgical material(s). The medical waste, regardless of its phase, is preferably collected so it neither fouls the surgical site nor becomes a biohazard in the medical suite in which the procedure is being performed. The medical waste may be removed from the surgical site through a suction tube under the influence of a vacuum provided by a suction source.

The suction source may be a vacuum pump on a medical waste collection system. An exemplary medical waste collection system is sold under the tradename Neptune by Stryker Corporation (Kalamazoo, Mich.). A manifold may provide a replaceable sterile barrier between the suction tube and the medical waste collection system. An unused manifold may be operably coupled with the medical waste collection system before or during the procedure, and the used manifold may be operably decoupled from the medical waste collection system during or after the procedure and discarded. The Neptune system also provides for “dumping” the waste material from an upper waste container to a lower waste container through a transfer valve. It is also contemplated to utilize a color camera to image the waste material within the waste container for estimating a concentration of hemoglobin within the waste material for blood loss determinations, as disclosed in commonly-owned International Publication No. WO 2023/177832, published Sep. 21, 2023, hereby incorporated by reference.

A common bodily fluid that is removed from a surgical site is the patient's blood. As the blood is removed from the surgical site, it passes through the suction tube, the manifold, and enters into the medical waste collection system where it is stored within the upper and/or lower waste container. Blood can coagulate, which is the action or process of a liquid, especially blood, changing to a solid or semi-solid state. Blood coagulation is commonly referred to as clotting. The clotting may occur within the upper waste container, which may complicate the dumping of the waste material through the transfer valve. In other words, the semi-solid or solid blood can clog the transfer valve. Moreover, the clots may result in portions of the waste material being redder in color, which may implicate the accuracy of the image-based determination of blood loss, which utilizes color component values of the image as disclosed in commonly-owned U.S. Pat. No. 8,792,693 , issued Jul. 29, 2014, the entire contents of which are hereby incorporated by reference.

Therefore, there is a need in the art to overcome one or more of the aforementioned shortcomings.

### SUMMARY

The subject disclosure is directed to an anticlotting element which serves as an anticoagulant which can help alleviate the potential clotting within the manifold and within the waste container(s). At least one anticlotting element can be placed within different parts of the manifold. The subject disclosure also provides for a method of using an anticlotting agent as an anticoagulant within the waste container(s). The subject disclosure also provides for an anticlotting assembly to be used in between the connection of the suction tube and the manifold.

According to a first aspect, the disclosure includes a manifold for a medical waste collection system, where the manifold includes a trunk, a head, at least one inlet fitting, and an anticlotting element. The head is coupled to the trunk and includes at least one inlet fitting. The inlet fitting is configured to removably receive a suction tube. The head defines a cavity and the anticlotting element is disposed within the cavity of the head.

According to a second aspect, the disclosure also provides a manifold for a medical waste system including a manifold receiver. The manifold includes a trunk, a head, at least one inlet fitting, a filter element, and an anticlotting element. The head is coupled to the trunk and includes at least one inlet fitting. The inlet fitting is configured to removably receive a suction tube. The trunk defines an outlet opening. The filter element is disposed within the trunk and the anticlotting element is disposed within the trunk.

According to a third aspect, the disclosure also provides a manifold for a medical waste collection system including a trunk. The trunk includes a body portion, a first leg, and a second leg. The manifold also includes a head, at least one inlet fitting, and an anticlotting element. The first leg extends from the body portion and defines an outlet opening. The second leg extends from the body portion and is spaced apart from the first leg to define a void. The head is coupled to the trunk. The inlet fitting is configured to removably receive a suction tube. The anticlotting element is disposed within the second leg of the trunk.

According to a fourth aspect, the disclosure also provides a manifold for a medical waste collection system. The manifold includes a trunk, a head, an inlet fitting, a tray, and an anticlotting element. The trunk defines an outlet opening and is coupled to the head. The head includes an accessory sleeve, and an inlet fitting which is coupled to the accessory sleeve. The inlet fitting is configured to be removably coupled with a suction tube. The coupling between the inlet fitting and the suction tube defines a suction path through the inlet fitting and through the trunk to the outlet opening. The tray defines a cavity with porous features configured to retain a tissue sample within the suction path. The tray is configured to be removably positioned within the accessory sleeve with the cavity opening toward an inlet bore. The anticlotting element is disposed within the cavity of the tray.

According to a fifth aspect, the disclosure also provides an anticlotting assembly for use with a medical waste collection system configured to receive a manifold. The anticlotting assembly includes a conduit. The conduit includes a distal fitting and a proximal fitting opposite the male fitting. The distal fitting is configured to be removably coupled to a suction tube or a suction instrument. The proximal fitting is configured to be removably coupled to an inlet fitting of the manifold. A chamber coupled to the conduit between the distal fitting and the proximal fitting. An anticlotting element is disposed within the chamber.

According to a sixth aspect, the disclosure also provides a method of docking a medical waste collection system with a docking station. The method includes effectuating removable coupling between the medical waste collection system and the docking station to establish an emptying fluid pathway and a cleaning fluid pathway. The method further includes operating the docking station to transfer medical waste being held on a waste container of the medical waste collection system to the docking station through the emptying fluid pathway. The method further includes operating the docking station to transfer a mixture of detergent and an anticlotting agent through the cleaning fluid pathway and prefilling the waste container of the medical waste collection system with the mixture of the detergent and the anticlotting agent.

Any of the above aspects can be combined in part or in whole with any other aspect. Any of the above aspects, whether combined in part or in whole, can be further combined with any of the following implementations, in full or in part.

### BRIEF DESCRIPTION OF THE DRAWINGS

Advantages of the present disclosure will be readily appreciated as the same becomes better understood by reference to the following detailed description when considered in connection with the accompanying drawings.

FIG. 1 is a perspective view of a medical waste collection system, and a docking station. A manifold is configured to be removably inserted into a receiver. An optional anticlotting assembly is shown as configured to be coupled to the manifold.

FIG. 2 is an exploded view of an implementation of the manifold.

FIG. 3 is an elevation view of a head of the manifold.

FIG. 4 is a side elevation view of the manifold with an anticlotting element positioned within the head.

FIG. 5 is a side elevation view of the manifold with an anticlotting element positioned within a filter element.

FIG. 6 is a side elevation view of the manifold with an anticlotting element positioned within a first leg of a trunk.

FIG. 7 is a side elevation view of the manifold with an anticlotting element positioned within the second leg of the trunk.

FIG. 8 is a perspective view of another implementation of the manifold in which a tray is removable couplable with an accessory sleeve. An anticlotting element may be positioned within the tray.

FIG. 9 is a top rear perspective view of the tray with the anticlotting element placed in multiple locations within the cavity of the tray.

### DETAILED DESCRIPTION

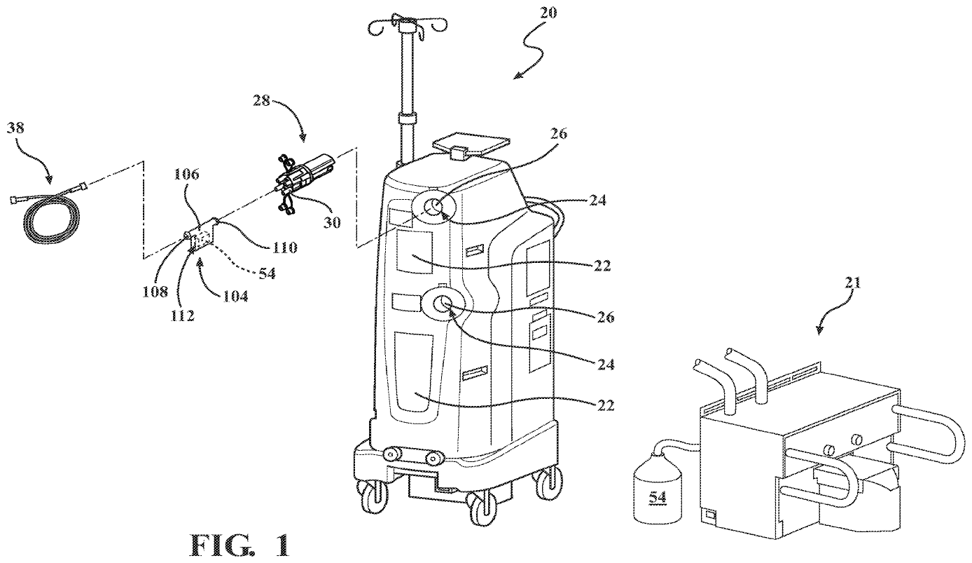

FIG. 1 shows a medical waste collection system 20 for collecting waste material generated during medical procedures or surgical procedures. The medical waste collection system includes at least one waste container 22 defining a waste volume for collecting and storing the waste material. The medical waste collection system 20 may also include a vacuum pump which is configured to draw suction on one or both of the first and second waste containers 22 through one or more vacuum lines. At least one manifold receiver 24 defines an opening 26 that is sized to removably receive at least a portion of a manifold 28 to be described throughout the present disclosure. FIG. 1 shows the medical waste collection system 20 with two manifold receivers 24; however, it should be appreciated that the medical waste collection system 20 may include a different number of receivers 24 than shown. Suitable construction and operation of several subsystems of the medical waste collection system 20 are disclosed in commonly-owned United States Patent Publication No. 2005/0171495, published Aug. 4, 2005, International Publication No. WO 2007/070570, published Jun. 21, 2007, International Publication No. WO 2014/066337, published May 1, 2014, International Publication No. WO 2017/112684, published Jun. 29, 2017, and the aforementioned International Publication No. WO 2020/210763, the entire contents of each being hereby incorporated by reference.

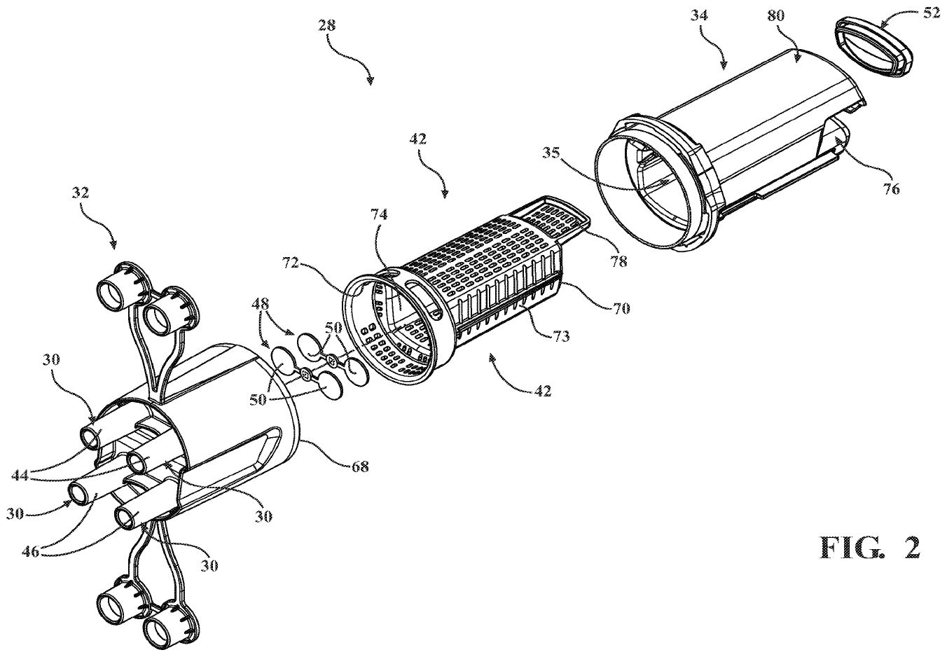

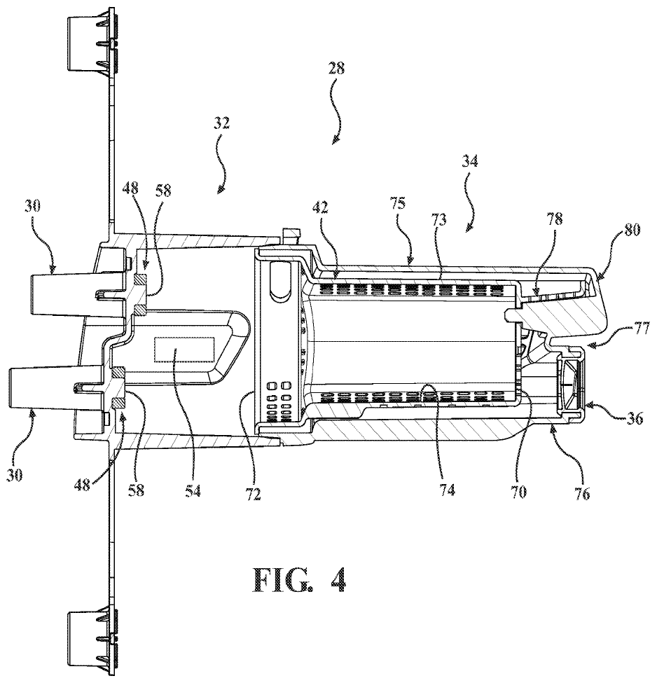

FIG. 2 shows an exploded view of the manifold 28. The manifold includes a head 32, and a trunk 34 coupled to the head 32. With further reference to FIG. 4, the trunk 34 may include a body portion 75, a first leg 76 extending proximally from the body portion 75, and a second leg 80 spaced apart from the first leg 76 by a void 77. The second leg 80 may extend proximally from the body portion 75. The first leg 76 of the trunk 34 defines an outlet opening 36, and a seal 52 may be coupled to a rim of the first leg 76 to cover the outlet opening 36. The head 32 includes the at least one inlet fitting 30 configured to removably receive a suction tube 38. The inlet fitting(s) 30 define an inlet bore 56 that open into a cavity 40 defined by the head 32. The inlet fittings 30 may include an upper pair of inlet fittings 44 and a lower pair of inlet fittings 46, corresponding to an upper pair of inlet bores 56 and a lower pair of inlet bores 56. At least one backflow prevention valve 48 may be coupled to the head 32 and disposed within the cavity 40. The illustrated implementation shows an upper backflow prevention valve 48 in which flappers 50 are configured to selectively cover the inlet bores 56 associated with the upper pair of inlet fittings 44, and a lower backflow prevention valve 48 in which flappers 50 are configured to selectively cover the inlet bores 56 associated with the upper pair of inlet fittings 46. The backflow prevention valves 48 may be coupled to couplers 58 disposed within the cavity 40 of the head 32.

A filter element 42 may be disposed within the trunk 34. The filter element 42 may include a tray 78, which extends to be within the second leg 80 of the trunk 34. The filter element 42 may include a proximal base 70, and a sidewall 73 that extends distally from the proximal base 70 to define a mouth 72 of the filter element 42. The filter element 42 may be shaped as a filter basket. Further aspects of the manifold 28 and the filter element 42 are disclosed in commonly-owned International Publication No. WO2020/209898, published Oct. 15, 2020, the entire contents of which are hereby incorporated by reference.

The anticlotting element 54 may include a substrate, and anticlotting agent. The substrate may take on any suitable form, for example, loose powder, a pod comprising loose powder within a dissolvable membrane, a dissolvable tablet, liquid, or the like. For example, the substrate could be in liquid form at the outset or pre-dissolved into a liquid concentrate from a dissolvable powder or a tablet. The anticlotting agent may include, in any suitable concentration or quantity, ethylenediaminetetraacetic acid (EDTA) and/or other compounds having anticlotting properties. EDTA is a well-known anticoagulant which inhibits clotting by binding the calcium ions within the blood. The concentration of EDTA within the anticlotting agent may be within any suitable range, and adjusted based on the type of surgical equipment and the kind of medical procedure being performed and the concentration of EDTA within the anticlotting agent can be beneficial. The illustrations of the substrate of the anticlotting element 54 in the figures are merely representative, and the substrate need not be box-shaped but rather may assume any suitable geometry, dimensions, form factor, and the like.

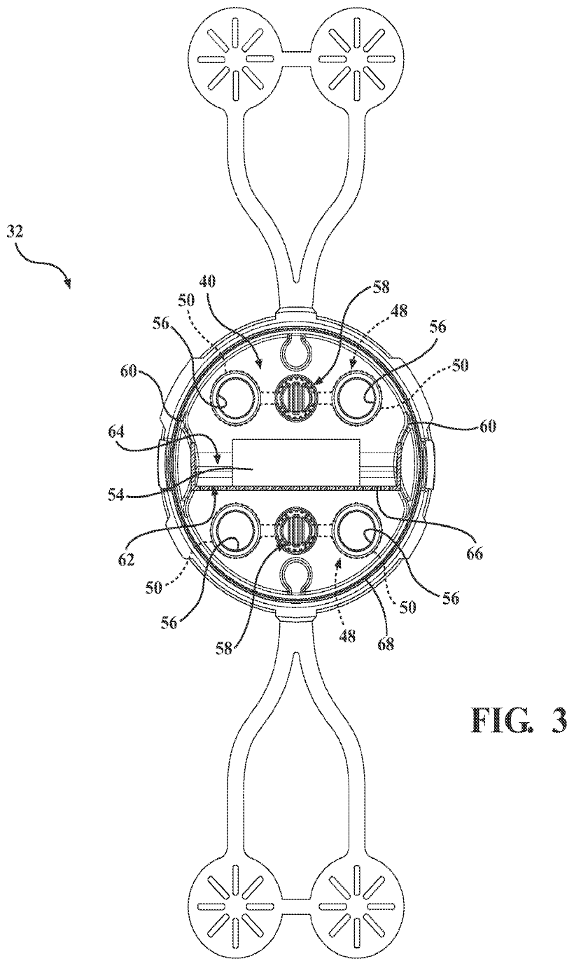

The anticlotting element 54 may be placed within any one or more locations within the manifold 28 as to be further described herein. Further, there may be one, two, three or more anticlotting elements 54, and the locations described herein may be used in combination. Referring first to FIGS. 3 and 4, the anticlotting element 54 may be disposed within the cavity 40 of the head 32 of the manifold 28. The anticlotting element 54 may be positioned proximal to the inlet bores 56, and thus proximal to the backflow prevention valves 48. Further, the anticlotting element 54 may be positioned axially between the backflow prevention valves 48 and the mouth 72 of the filter element 42.

The backflow prevention valves 48 are designed to open as the vacuum draws medical waste through the inlet bores 56 to the waste containers 22. If the anticlotting element 54 prevents the backflow prevention valves 48 from opening, then a situation could arise where the medical waste is not properly suctioned through the manifold 28 into the waste container(s) 22. Therefore, in certain implementations, the anticlotting element 54 may be positioned, in elevation, between the backflow prevention valves 48. Doing so provides at least two advantages. First, the flappers 50 of the backflow prevention valves 48 are able to flex proximally or inwardly in an unimpeded manner under the influence of suction being drawn through the manifold 28. Likewise, the fluid flow entering the internal volume of the manifold 28 is unobstructed. In other words, the fluid being drawn into the internal volume through the upper pair of the inlet fittings 44 may pass along an upper aspect or surface of the anticlotting element 54, and the fluid being drawn into the internal volume through the lower pair of the inlet fittings 46 may pass along a lower aspect or surface of the anticlotting element 54. The fluid flow is generally unobstructed, yet interaction occurs between the fluid flow and the anticlotting element 54 for the anticlotting element 54 to dissolve, mix, or the like, which is then suctioned with the fluid flow into the waste container(s) 22 of the medical waste collection system 20. For example, in instances where the anticlotting element 54 is a dissolvable tablet, frictional forces from the incoming fluid flow may slowly dissolve upper and lower surfaces of the tablet without meaningfully affecting the flow rate of the fluid flow through the manifold 28.

FIG. 3 shows an exemplary manner by which the anticlotting element 54 may be supported within the manifold 28 to be positioned between the backflow prevention valves 48. The head 32 of the manifold 28 may include ribs 60 or other suitable geometries coupled to an inner surface of the head 32. The longitudinal ribs 60 may be opposing pairs of ribs. The manifold 28 may also include a support structure 62 which is supported by the ribs 60. The support structure 62 may be removably or fixedly coupled to the ribs 60. For example, the support structure 62 may be a cage or tray configured to be slidably inserted between the ribs 60 during assembly of the manifold 28. Alternatively, the support structure 62 need not be a discrete component, but rather integrally formed with the head 32 during manufacturing of the manifold 28. In such an alternative arrangement, the ribs 60 are optional.

The support structure 62 may define a cavity 64 and porous features 66. The cavity 64 may be sized and shaped to conform to the form factor of the anticlotting element 54. For example, upper, lower, and/or lateral walls or barriers may be sized to limit shifting or jostling of the anticlotting element 54 within the support structure 62 against forces from the incoming fluid flow. As best shown in FIG. 3, the anticlotting element 54 may be disposed within the cavity 64 of the support structure 62. During, for example, the dissolving of the anticlotting element 54, the fluid flow with the anticlotting agent mixed therein, can pass through the porous features 66. The porous features 66 may be sized to ensure the anticlotting element 54 is sufficiently dissolved before being permitted to pass therethrough and through the manifold 28. In another implementation, the anticlotting element 54 may be directly supported within the head 32. For example, the substrate may be a tablet that is sized to be supported by the ribs 60 or other suitable geometries.

FIG. 4 is intended to represent the anticlotting element 54 being positionable in any suitable location within the cavity 40. In certain implementations, the filter element 42 may be modified to facilitate supporting the anticlotting element 54 in its desired location. For example, the anticlotting element 54 could be placed where it is disposed distal to the mouth 72 of the filter element 42. The filter element 42 may include geometries or support features (e.g., a crossbeam) configured to engage a proximal aspect of the substrate of the anticlotting element 54. In other words, the anticlotting element 54 may be axially compressed between the filter element 42 and a counterpart surface, structure, or feature of the head 32 to maintain an axial position of the anticlotting element 54 within the cavity 40.

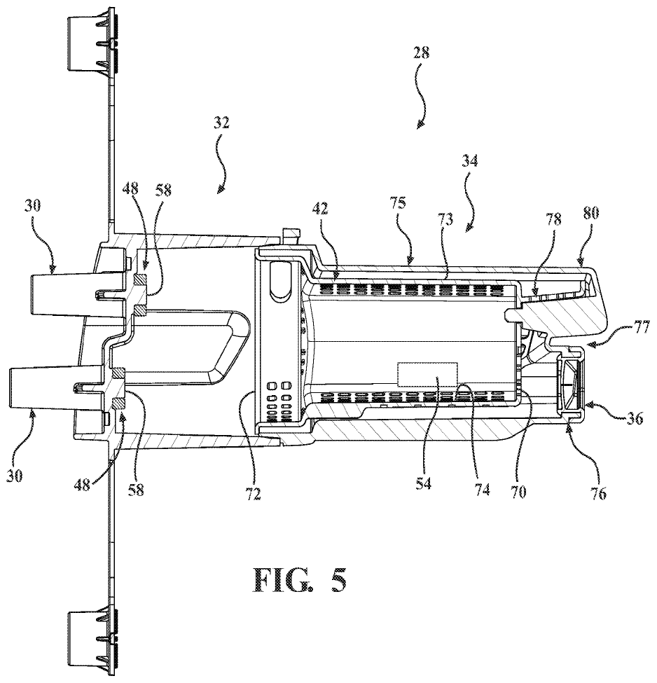

FIGS. 5-7 show the anticlotting element 54 positioned at various locations within the trunk 34 of the manifold 28. FIG. 5 shows the anticlotting element 54 being disposed within the filter element 42. In other words, the substrate is positioned between the proximal base 70 and the mouth 72 of the filter element 42. The substrate may be coupled to an inner surface 74 of the filter element 42, or free-floating within the same. The latter may include the substrate merely resting against a lower aspect of the sidewall and against the proximal base 70 of the filter element 42. To the extent the anticlotting element 54 shifts during handling and coupling of the manifold 28 into the medical waste collection system 20, the incoming fluid flow being drawn through the manifold 28 may merely urge it again into contact with the proximal base 70. In certain implementations, the filter element 42 may include geometries, such as projections, protrusions, barriers, or the like, against which the anticlotting element 54 is disposed to maintain the position of the anticlotting element 54 relative to the filter element 42. For example, the filter element 42 may be integrally formed with barriers having porous features to define a cavity, and the substrate of the anticlotting element 54 may be a dissolvable tablet disposed within the cavity. Placing the anticlotting element 54 within the filter element 42 has the benefit of preventing or limiting clotting within the filter element 42, improving the operational life of the manifold 28. In addition to the anticlotting of the waste material within the waste containers 22, in this respect the anticlotting element 54 has a dual purpose.

The anticlotting element 54 may also be placed in different parts of the trunk 34 without being placed within the filter element 42. For example, the filter element 42 may be narrower in width than shown in FIG. 5, wherein the anticlotting element 54 is positioned or coupled outside of the filter element 42 but within the trunk 34.

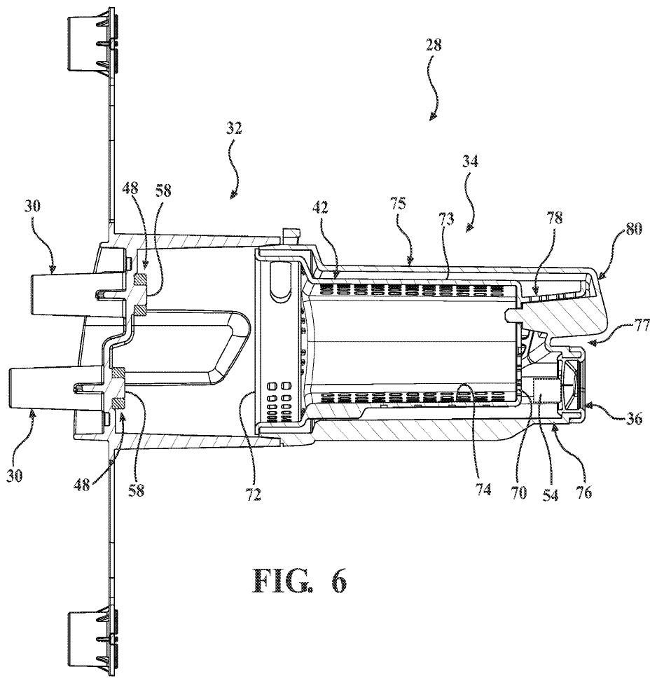

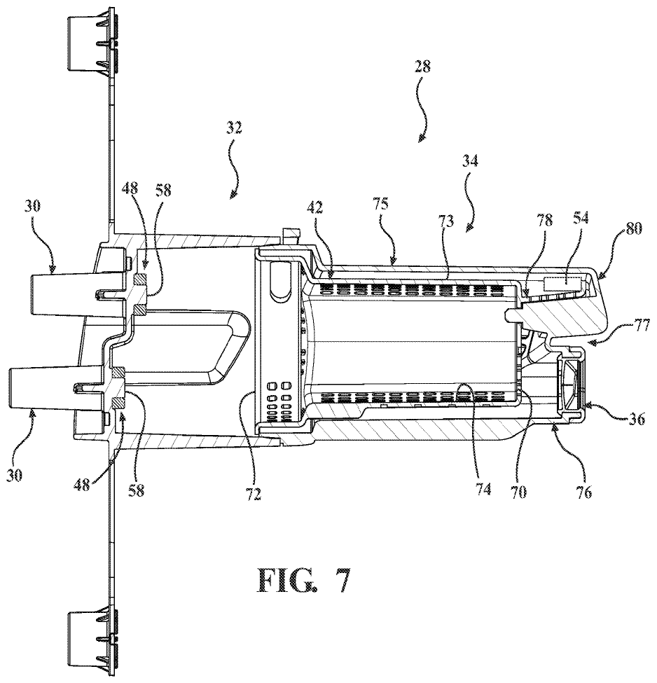

For another example, FIG. 6 shows the anticlotting element 54 may be placed downstream of the filter element 42, and between the proximal base 70 and the outlet opening 36. In such an arrangement, the anticlotting element 54 is positioned within the first leg 76 of the trunk 34. FIG. 7 shows another arrangement in which the anticlotting element 54 is within the second leg 80 of the trunk 34. In implementations in which the filter element 42 includes the tray 78, the anticlotting element 54 may be supported by the tray 78. In implementations in which the filter element 42 does not include the tray, the anticlotting element 54 may be retained within the second leg 80 by the proximal base 70 of the filter element 42. The substrate of the anticlotting element 54 may be formed to be contoured to dimensions of an inner surface of the second leg.

In another implementation, the substrate of the anticlotting element 54 may be loose powder, fill, or granules. In such an implementation, a method of assembly of the manifold 28 may include supporting the trunk in a generally vertical orientation. For example, the trunk may be placed in a jig or clamp with the opening to within the manifold volume 35 facing generally upwardly. The substrate of the anticlotting element may be directed into the second leg under the influence of gravity. In other words, the anticlotting element 54 may be poured into the manifold volume 35. The substrate may be compacted within the second leg 80. In one example, a plunger device (not shown) may be used to compact the substrate within the second leg 80. The filter element 42 may be inserted into the manifold volume 35, and coupled to the trunk 34. The head 32 may also be coupled to the trunk 34.

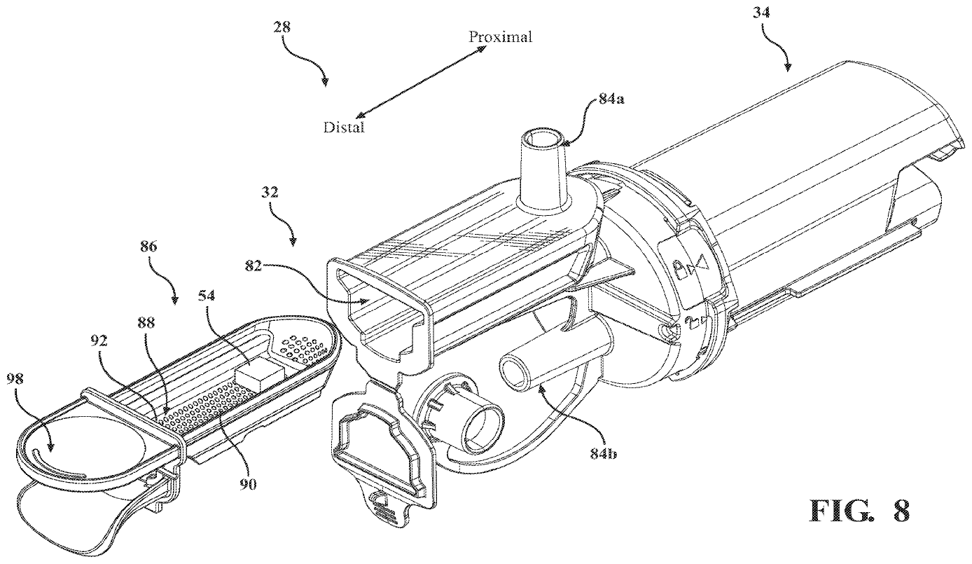

Referring now to FIG. 8, another implementation of the manifold 28 is shown in which a tray 86 may be used to collect tissue samples, or for supporting the anticlotting element 54. The manifold 28 may be used to conveniently alternate between these functions in a relatively seamless manner. The head 32 includes an accessory sleeve 82, with the inlet fitting 84a coupled to the accessory sleeve 82. The inlet fitting 84a of the accessory sleeve 82 is configured to be removably coupled with the suction tube 38. The coupling between the inlet fitting 84a and the suction tube 38 defines a suction path through the inlet fitting 84a and through the trunk 34 to the outlet opening 36. The trunk 34 may generally be as previously described.

The tray 86 includes a base 92 and a sidewall defining a cavity 88 and porous features 90 configured to retain an object with the suction path (e.g., the tissue sample or the anticlotting element 54) without overly impeding the fluid flow. The tray 86 is configured to be removably positioned within the accessory sleeve 82 with the cavity 88 opening towards the inlet bore 56. Certain additional features of the tray 86 are disclosed in commonly-owned United States Patent Publication No. 2021/0162101, published Jun. 3, 2021, the entire contents of which are hereby incorporated by reference.

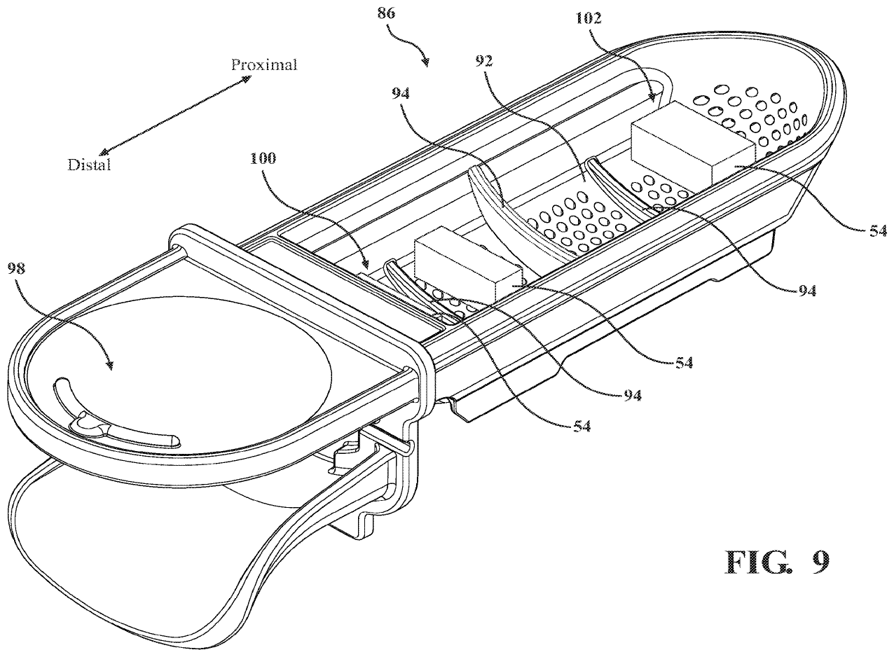

As seen in FIG. 8, the anticlotting element 54 is disposed within the cavity 88 of the tray 86. With concurrent reference to FIG. 9, the tray 86 may include a barrier 94 that divides the cavity 88 into compartments 96. The tray 86 may also include a distal handle 98, wherein the compartments 96 include a distal compartment 100 adjacent to the distal handle 98 and a proximal compartment 102 aligned with the inlet fitting 30. The anticlotting element 54 may be disposed within one of the compartments 96 including the distal compartment 100 and/or the proximal compartment 102. FIG. 9 further shows the tray 86 including the barrier 94 extending upwardly from the base 92 to a distance less than a height of the cavity 88 of the tray 86.

In certain instances, it may be desirable for the anticlotting element 54 to be on a separate component. For example, the suction system may use a freestanding canister with a wall-based vacuum source as opposed to the medical waste collection system 20. Alternatively, the user may have manifolds operable with the medical waste collection system 20, but not versions of the manifold that include the anticlotting element 54. Therefore, objects of the present disclosure may be incorporated into an anticlotting assembly 104 that is configured to be coupled with opposing suction tubes to be coupled to the freestanding canister, or coupled via suction tubes or directly to the inlet fitting 30 of the manifold.

The anticlotting assembly 104 includes a conduit 106. The conduit 106 includes a distal fitting 108, a proximal fitting 110 opposite the distal fitting 108, and a chamber 112. The distal fitting 108 is configured to be removably coupled to the suction tube 38. The proximal fitting 110 is configured to be removably coupled to the inlet fitting 30 of the manifold 28, or another suction tube, as mentioned. The chamber 112 is in fluid communication with the conduit 106. In certain implementation, the conduit 106 and the chamber 112 may include complimentary coupling features such that the chamber 112 is removably coupled to the conduit 106 so as to function as a cartridge.

Medical waste is collected using the vacuum pump to pull medical waste through the suction tube 38, the anticlotting assembly 104, the manifold 28 and into the waste container(s) 22. The anticlotting element 54 may be disposed within the chamber 112 of the conduit 106 of the anticlotting assembly 104. As the medical waste is removed from the surgical site, it passes through the anticlotting assembly 104 and through the chamber 112. The anticlotting element 54 interacts with the fluid flow passing through the conduit 106. For example, the substrate may be a loose powder within a dissolvable membrane or a dissolvable tablet, which is drawn into the fluid flow.

The anticlotting assembly 104 can also be arranged where the chamber 112 is configured to be suspended from the conduit 106 such that the anticlotting element 54 is siphoned from the chamber 112 to within the conduit 106. The anticlotting assembly 104 may further include an orientation feature coupled to one of the conduit 106 and the chamber 112 and configured to require the anticlotting assembly 104 be coupled to the manifold 28 in a single rotational orientation in which the chamber 112 is suspended from the conduit 106. In another variant, the conduit 106 and the chamber 112 are separated by the dissolvable membrane. The dissolvable membrane allows for a temporary barrier which will keep the anticlotting element 54 separated from the rest of the anticlotting assembly 104 until removal of medical waste is necessary.

Additionally or alternatively, it is further contemplated that the anticlotting agent may be provided through a docking protocol between the medical waste collection system 20 and a docking station 21 (see FIG. 1). The medical waste collection system 20 is configured to be removably coupled to the docking station 21, after which an emptying fluid pathway and a cleaning fluid pathway is established. The waste material within the waste containers 22 is emptied, and a mixture of water and detergent is transferred to the medical waste collection system 20. In particular, water or the mixture may be prefilled within the upper waste container 22 to set a zero point for a fluid measuring subsystem of the medical waste collection system 20. Further details regarding the docking station and prefilling is disclosed in commonly-owned International Publication No. WO2007/070570, published Jun. 21, 2007, the entire contents of which are hereby incorporated by reference.

FIG. 1 shows a reservoir of the anticlotting element 54 in fluid communication with the docking station 21. The anticlotting element 54 may be mixed with the detergent, or a standalone reservoir. A method of docking the medical waste collection system 20 includes effectuating removable coupling between the medical waste collection system 20 and the docking station to establish the emptying fluid pathway and the cleaning fluid pathway.

The method further includes operating the docking station to transfer medical waste being held in the waste container(s) 22 of the medical waste collection system 20 to the docking station through the emptying fluid pathway. The method may include receiving an input from a user that is based on a subsequent medical procedure being at a greater likelihood of clotting of blood within the waste container(s) of the medical waste collection system. The method further includes operating the docking station to transfer a mixture of a detergent and the anticlotting agent through the cleaning fluid pathway and prefilling the waste container of the medical waste collection system with the mixture of the detergent and the anticlotting agent. In view of the foregoing devices and methods, clotting within the waste containers 22 is minimized or eliminated. As a result, once the upper waste container 22 is sufficiently full and it is desired to be dumped through the transfer valve to the lower waste container, the likelihood of clogging of the transfer valve is minimized. Further,

Several implementations have been discussed in the foregoing description. However, the implementations discussed herein are not intended to be exhaustive or limit the disclosure to any particular form. The terminology which has been used is intended to be in the nature of words of description rather than of limitation. Many modifications and variations are possible in light of the above teachings and the disclosure may be practiced otherwise than as specifically described. It should be appreciated that the anticlotting element 54 may include more than one anticlotting element 54 disposed in various locations within the trunk 34 and/or the head 32 according to embodiments disclosed herein.

1. A manifold for a medical waste collection system, the manifold comprising:
a trunk defining an outlet opening;
a head coupled to the trunk and comprising at least one inlet fitting configured to removably receive a suction tube, wherein the head defines a cavity; and
an anticlotting element disposed within the cavity of the head, wherein the anticlotting element comprises a substrate, and anticlotting agent.

2. The manifold of claim 1, further comprising a filter element disposed within the trunk, wherein the anticlotting element is disposed distal to a mouth of the filter element.

3. The manifold of claim 2, wherein opposing sides of the mouth of the filter element engages the anticlotting element to maintain an axial position of the anticlotting element within the cavity.

4. The manifold of claim 1, wherein the at least one inlet fitting comprises an upper pair of inlet fittings and a lower pair of inlet fittings, and wherein the anticlotting element is positioned proximal to and between inlet bores defined by the upper and lower pairs of inlet fittings.

5. The manifold of claim 1, further comprising at least one backflow prevention valve coupled to the head and disposed within the cavity, wherein the anticlotting element is disposed proximal to the backflow prevention valve.

6. The manifold of claim 5, wherein the at least one inlet fitting comprises an upper pair of inlet fittings and a lower pair of inlet fittings, wherein the at least one backflow prevention valve comprises an upper backflow prevention valve configured to selectively cover inlet bores of the upper pair of inlet fittings, and a lower backflow prevention valve configured to selectively cover inlet bores of the lower pair of inlet fittings, and wherein the anticlotting element is positioned between the upper and lower backflow prevention valves.

7. The manifold of claim 1, wherein the head comprises longitudinal ribs coupled to an inner surface of the head, wherein the anticlotting element is supported by the ribs.

8. The manifold of claim 7, wherein the longitudinal ribs further comprise opposing pairs of ribs, wherein the anticlotting element is supported between the opposing pairs of ribs.

9. The manifold of claim 7, further comprising a support structure supported by the ribs and defining a cavity and porous features, wherein the anticlotting element is disposed with the support structure.

10-13. (canceled)

14. A manifold for a medical waste collection system, the manifold comprising:
a trunk comprising a body portion, a first leg extending from the body portion and defining an outlet opening, and a second leg extending from the body portion and spaced apart from the first leg to define a void;
a head coupled to the trunk and comprising at least one inlet fitting configured to removably receive a suction tube; and
an anticlotting element disposed within the second leg of the trunk, wherein the anticlotting element comprises a substrate, and anticlotting agent.

15. The manifold of claim 14, further comprising a filter element disposed with the trunk and comprising a tray extending to within the second leg, wherein the anticlotting element is supported by the tray of the filter element.

16. The manifold of claim 15, further comprising a filter element disposed with the trunk and comprising a proximal base, and a sidewall extending from the proximal base to define a mouth, and wherein the anticlotting element is retained with the second leg by the proximal base of the filter element.

17. The manifold of claim 15, wherein the anticlotting element is formed to be contoured to dimensions of an inner surface of the second leg.

18. (canceled)

19. (canceled)

20. A manifold for a medical waste collection system, the manifold comprising:
a trunk defining an outlet opening;
a head coupled to the trunk and comprising an accessory sleeve, and an inlet fitting coupled to the accessory sleeve and configured to be removably coupled with a suction tube to define a suction path through the inlet fitting and through the trunk to the outlet opening;
a tray defining a cavity and porous features configured to retain a tissue sample within the suction path, wherein the tray is configured to be removably positioned within said accessory sleeve with the cavity opening towards said inlet fitting; and
an anticlotting element disposed within the cavity of the tray, wherein the anticlotting element comprises a substrate, and anticlotting agent.

21. The manifold of claim 20, wherein the tray further comprises a barrier that divides the cavity into compartments, wherein the anticlotting element is disposed within one of the compartments.

22. The manifold of claim 21, wherein the tray further comprises a distal handle, wherein the compartments comprise a distal compartment adjacent the distal handle, and a proximal compartment configured to be aligned with the inlet fitting, and wherein the anticlotting element is disposed within the proximal compartment.

23. The manifold of claim 21, wherein the tray further comprises a distal handle, wherein the compartments comprise a distal compartment adjacent the distal handle, and a proximal compartment configured to be aligned with the inlet fitting, and wherein the anticlotting element is disposed within the distal compartment.

24. The manifold of claim 21, wherein the barrier extends upwardly from a base to a distance less than a height of the cavity of the tray.

25. (canceled)

26. (canceled)

27. The manifold of claim 1, wherein the substrate is one of loose powder, a pod comprising loose powder within a dissolvable membrane, or a dissolvable tablet.

28-37. (canceled)
